(19) United States
(12) Patent Application Publication
(10) Pub. No.: US 2022/0047493 A1
(43) Pub. Date: Feb. 17, 2022

(54) COMPOSITION OF ACTIVE INGREDIENTS, FOR CARE OF HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); TORSTEN LECHNER, Langenfeld (DE)

(73) Assignee: Henkel AG & Co. KGaA, Duesseldorf (DE)

(21) Appl. No.: 17/290,203

(22) PCT Filed: Oct. 31, 2019
(86) PCT No.: PCT/EP2019/079788

§ 371 Date: Apr. 29, 2021

(30) Foreign Application Priority Data
Oct. 31, 2018 (DE) 10 2018 127 180.8

(51) Int. Cl. A61K 8/92; A61K 8/58; A61Q 5/12

(52) U.S. Cl. CPC A61K 8/922; A61Q 5/12; A61K 8/585

(57) ABSTRACT

The present invention relates to an active ingredient composition for the care and surface modification of human hair. In particular, the invention relates to a cosmetic composition for treating a keratinous material comprising a) at least one organic silicon compound and b) a sulfated and/or sulfonated fatty acid ester, the cosmetic composition being particularly suitable for caring for damaged hair.

### CROSS-REFERENCE TO RELATED APPLICATION

This application is a U.S. National-Stage entry under 35 U.S.C. § 371 based on International Application No. PCT/EP2019/079788, filed Oct. 31, 2019, which was published under PCT Article 21(2) and which claims priority to German Application No. 10 2018 127 180.8, filed Oct. 31, 2018, which are all hereby incorporated in their entirety by reference.

### TECHNICAL FIELD

The present disclosure relates to cosmetic compositions for treating a keratinous material, the composition comprising an organic silicon compound and a sulfated or sulfonated fatty acid ester, and the use of the cosmetic composition.

### BACKGROUND

The external exposure of hair to chemicals from a variety of different sources poses challenges for the development of cosmetic care products. Air and water impurities have a detrimental effect on skin and hair. Major air pollutants include polycyclic aromatic hydrocarbons, volatile organic compounds, nitrogen oxides (NOx), particulate matter, and cigarette smoke. The effect of various air pollutants can be enhanced in the presence of other air pollutants and when exposed to UV radiation.

It is known that the toxicity of gaseous pollutants in the air, such as sulfur dioxide, ozone, and nitrogen oxides, is related to their initiator activity for free radicals, which cause damage to living organisms. Free radicals are metabolic products that also occur naturally in the body. In large quantities, free radicals can promote irritation and inflammation and accelerate the process of aging. In this case, the term “oxidative damage” is used. Free radicals can also cause hair damage, which is visible, for example, as a reduction in shine as well as grip and/or fading of hair color.

Particulate matter is a complex mixture containing metals, minerals, organic toxins, and/or biological materials. They can also promote the formation of free radicals.

Furthermore, often changing consumer demands for a certain hair texture are associated with recurring chemical exposure of the hair. For example, hair coloring stresses the hair, due to which a special, intensive care may be necessary.

In the prior art, organosilicon compounds from the group of silanes comprising at least one hydroxy group and/or hydrolyzable group are described. Due to the presence of the hydroxy groups and/or hydrolyzable groups, the silanes are reactive substances that hydrolyze or oligomerize or polymerize in the presence of water. The oligomerization or polymerization of the silanes initiated by the presence of the water, when applied to a keratinous material, ultimately leads to the formation of a film that can exert a protective effect.

### BRIEF SUMMARY

In exemplary embodiments, a cosmetic composition is provided for treating a keratinous material. An exemplary cosmetic composition comprises at least one organic silicon compound containing one to three silicon atoms, and at least one sulfated and/or sulfonated fatty acid ester.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure is to provide a product with an improved care and/or protection effect. In particular, the present disclosure was based on the task of providing a cosmetic agent which, after a hair treatment, enables a caring after-treatment which provides special care to stressed hair.

This task is solved by a cosmetic agent for the treatment of a keratinous material, comprising:

a) at least one organic silicon compound containing one to three silicon atoms, and
b) at least one sulfated and/or sulfonated fatty acid ester.

By a keratinous material is meant hair, the skin, the nails (such as fingernails and/or toenails). Wool, furs, and feathers also fall under the definition of keratinous material.

Preferably, a keratinous material is understood to mean human hair, human skin, and human nails, in particular fingernails and toenails. Very preferably, keratinous material is understood to mean human hair, in particular head and/or beard hair.

As a first ingredient essential to the invention, the cosmetic composition for treating a keratinous material contains at least one organic silicon compound containing one to three silicon atoms. Preferred organic silicon compounds are selected from silanes having one, two or three silicon atoms, wherein the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolyzable groups per molecule.

In a most preferred embodiment, the agent for treating a keratinous material comprises at least one organic silicon compound selected from silanes having one, two or three silicon atoms, wherein the organic silicon compound further comprises one or more basic groups and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a Di(C₁-C₆)alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),
where

R₁, R₂ both represent a hydrogen atom,

L represents a linear, two-band C₁-C₆-alkylene group, preferably a propylene group (—CH₂—CH₂—CH₂—) or an ethylene group (—CH₂—CH₂—),

R₃, R₄ independently represent a methyl group or an ethyl group,

a stands for the number 3, and

b stands for the number 0;

(R₅O)_{c}(R₆)_{d}Si-(A)_{c}-[NR₇-(A′)]_{f}—[O-(A″)]_{g}-[NRs-(A′″)]ₕ-Si(R₆′)_{d′}(OR₅′)_{c′} (II),

where

R5, R5′, R5″ independently represent a hydrogen atom or a C₁-C₆ alkyl group,

R6, R6′ and R6″ independently represent a C₁-C₆ alkyl group,

A, A′, A″, A′″ and A″″ independently represent a linear or branched C₁-C₂₀ divalent alkylene group,

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1, and
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear two-band C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched two-band C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, two-band C₁-C₂₀ alkylene group.

Preferably, -L- represents a linear, divalent C₁-C₂₀ alkylene group. Further preferably, -L- represents a linear divalent C₁-C₆ alkylene group. Particularly preferred -L stands for a methylene group (—CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. R₃ and R₄ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are

(3-Aminopropyl)triethoxysilan

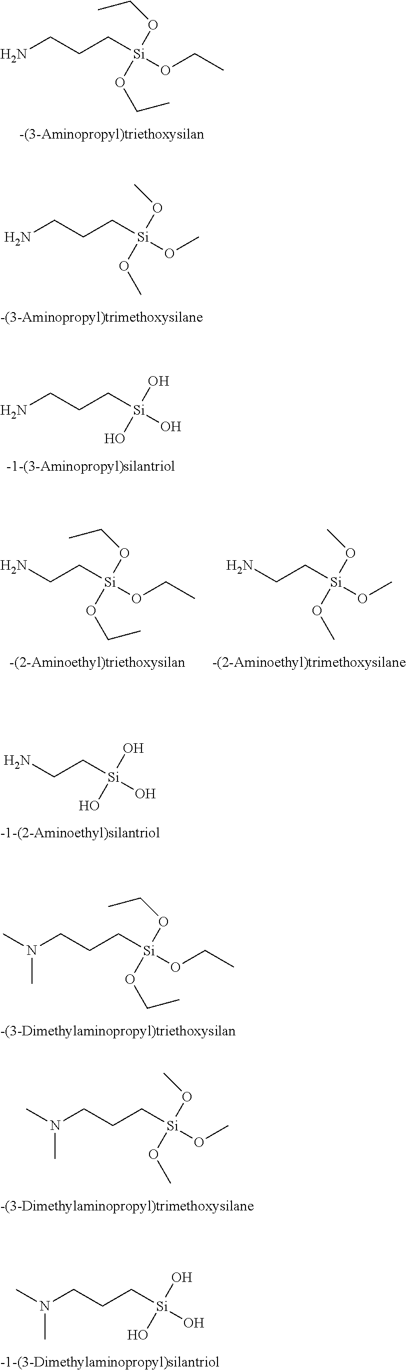

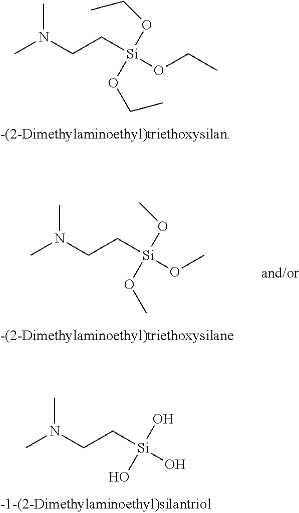

The organic silicon compound of formula (I) is commercially available. (3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material comprises at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR_{5′})_{c′} (II).

The organosilicon compounds of formula (II) each bear at their two ends the silicon-containing groupings (R₅O)_{c}(R₆)_{d}Si— and —Si(R₆′)_{d′}(OR_{5′})_{c′}.

In the central part of the molecule of formula (II) there are the groups -(A)_{c} and —[NR₇-(A′)]_{f}- and —[O-(A″)]_{g}- and -[NRs-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) contains at least one grouping selected from the group consisting of -(A)- and —[NR₇-(A′)]- and —[O-(A″)]- and —[NR₈-(A′″)]-.

In the two terminal structural units (R₅O)_{c}(R₆)_{d}Sii- and—Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

An extremely high anti-pollution effect of the agent for the treatment of a keratinous material could be obtained when the residues c and c′ both stand for the number 3. In this case d and d′ both stand for the number 0.

In another preferred one, the agent for treating a keratinous material comprises at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR_{5′})_{c′} (II),

where

R5 and R5′ independently represent a methyl group or an ethyl group,

c and c′ both stand for the number 3, and

d and d′ both stand for the number 0.

When c and c′ both represent the number 3 and d and d′ both represent the number 0, the organic silicon compounds correspond to formula (IIa)

(R₅O)₃Si-(A)_{c}-[NR₇-(A′)]_{f}—[O-(A″)]_{g}—[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and —[NR₇-(A′)]_{f}- and —[O-(A″)]_{g}- and -[NR8-(A′″)]ₕ- are in the middle part of the organic silicon compound of formula (II).

In this context, the presence of certain groupings has proven to be particularly beneficial in terms of increasing the “anti-pollution” effect. Particularly good results were obtained when at least two of the residues e, f, g, and h stand for the number 1. Especially preferred e and f both stand for the number 1. Furthermore, g and h both stand for the number 0.

When e and f are both 1 and g and h are both 0, the organic silicon compounds are represented by the formula (IIb)

(R₅O)_{c}(R₆)_{d}Si-(A)-[NR₇-(A′)]-Si(R₆′)_{d′}(OR₅′)_{c}, (IIb).

The radicals A, A′, A″, A′″ and A″″ independently represent a linear or branched two band C₁-C₂₀ alkylene group. Preferably the radicals A, A′, A″, A′″ and A″″ independently of one another represent a linear, two band C₁-C₂₀ alkylene group. Further preferably the radicals A, A′, A″, A′″ and A″″ independently represent a linear two band C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR₇-(A′)]-.

When the radical h represents the number 1, the organic silicon compound of formula (II) contains a structural grouping —[NR₈-(A″)]-.

Wherein R₇ and R₇ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)-Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

When the radical f represents the number 1 and the radical h represents the number 0, the organic silicon compound contains the grouping [NR₇-(A′)] but not the grouping —[NRs-(A″)]. If the radical R₇ now stands for a grouping of the formula (III), the agent for treating a keratinous material contains an organic silicon compound with 3 reactive silane groups.

In another preferred one, the agent for treating a keratinous material comprises at least one organic silicon compound of formula (II)

(R₅O)_{c}(R₆)_{d}Si-(A)_{c}-[NR₇-(A′)]_{f}—[O-(A″)]_{g}—[NR₈-(A′″)]ₕ-Si(R₆′)_{d′}(OR₅′)_{c′} (II),

where

e and f both stand for the number 1,

g and h both stand for the number 0,

A and A′ independently represent a linear, two-band C₁-C₆ alkylene group, and

R7 represents a hydrogen atom, a methyl group, a 2-hydroxyethyl group, a 2-alkenyl group, a 2-aminoethyl group or a group of formula (III).

In another preferred embodiment, the composition for treating a keratinous material comprises at least one organic silicon compound of the formula (II), wherein

e and f both stand for the number 1,

g and h both stand for the number 0,

A and A′ independently of one another represent a methylene group (—CH₂—), an ethylene group (—CH₂—CH₂—) or a propylene group (—CH₂—CH₂—CH₂—),

and

R₇ represents a hydrogen atom, a methyl group, a 2-hydroxyethyl group, a 2-alkenyl group, a 2-aminoethyl group or a group of formula (III).

Organic silicon compounds of formula (II) that are well suited for solving the problem are

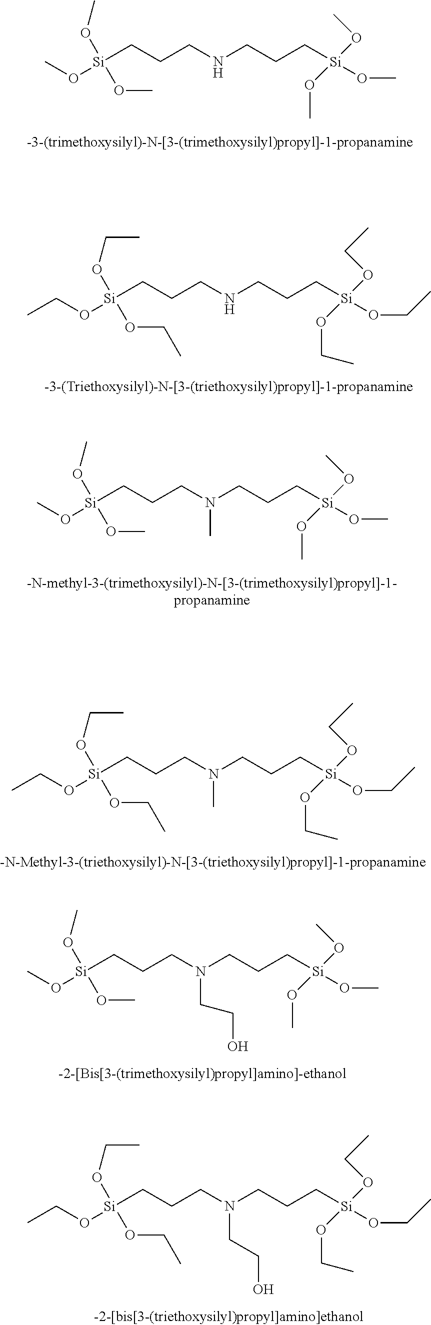

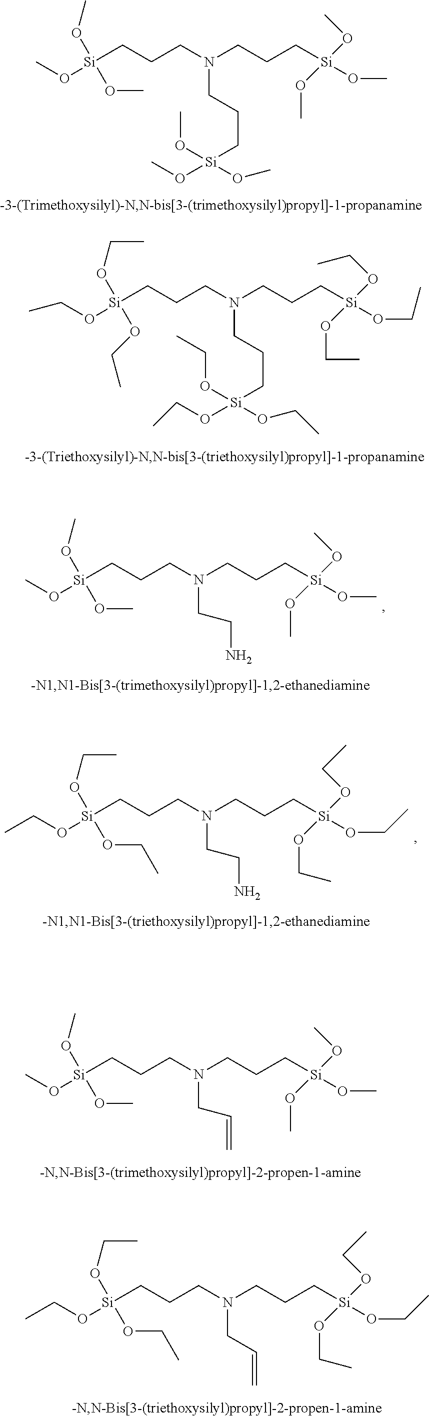

The organic silicon compounds of formula (II) are commercially available.

Bis(trimethoxysilylpropyl)amine with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, with CAS number 13497-18-2 kcan be purchased, for example, from Sigma-Aldrich or is commercially available under the product name Dynasylan 1122 from Evonik.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous when the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further preferred embodiment, the composition for treating a keratinous material contains, in addition to the organic silicon compound or compounds of formula (I), at least one further organic silicon compound of formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a likewise preferred embodiment, the composition for treating a keratinous material contains, in addition to the organic silicon compound or compounds of the formula (II), at least one further organic silicon compound of the formula (IV) contains

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In another preferred embodiment, the composition for treating a keratinous material contains, in addition to the organic silicon compounds of the formula (I) and (II), at least one further organic silicon compound of the formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In the organic silicon compounds of formula (IV), the radical R₉ represents a C₁-C₁₂ alkyl group. This C₁-C₁₂ alkyl group is saturated and can be linear or branched. Preferably R₉ stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. Particularly preferably, R₁₀ represents a methyl group or an ethyl group.

In the organic silicon compounds of formula (IV), the radical R₁₁ represents a C₁-C₆ alkyl group. Particularly preferably, R₁₁ represents a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

An extremely high “anti-pollution” effect could be obtained if the agent for treating a keratinous material contains at least one organic silicon compound of formula (IV) in which the radical k represents the number 3. In this case the rest m stands for the number 0.

Organic silicon compounds of the formula (IV) that are particularly suitable for solving the problem are

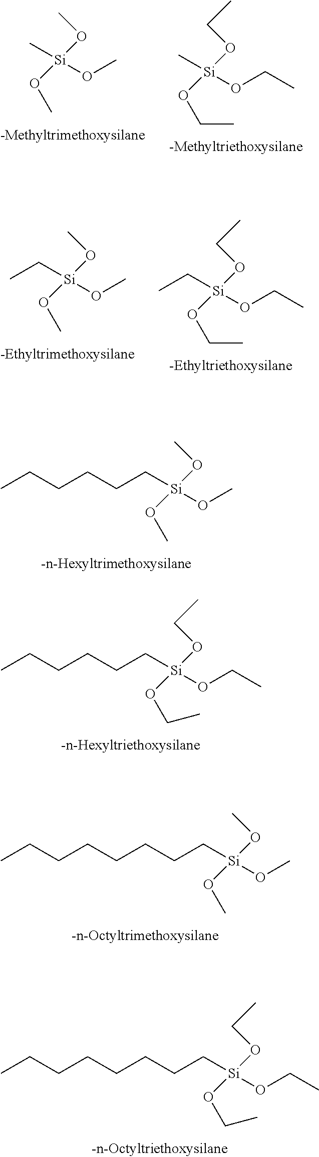

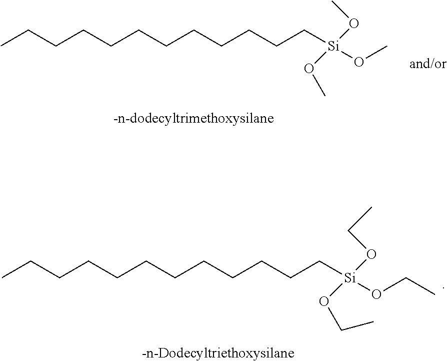

and propyltrimethoxysilane, propyltriethoxysilane, octadecyltrimethoxysilane and/or octadecyltriethoxysilane.

The organic silicon compounds described above are reactive compounds.

In this context, it has been found to be quite preferred if the agent contains as organic silicon compound (3-aminopropyl)triethoxysilane, i.e., an aminopropyltriethoxysilane (AMEO), and/or 3-(triethoxysilyl)-N-[3-(triethoxysilyl)propyl]-1-propanamine, i.e., a bis(triethoxysilylpropyl)amine.

According to preferred embodiments, the organic silicon compound of formula (I), in particular the (3-aminopropyl)triethoxysilane, is present in an amount of from about 0.01 to about 10 w % by weight, preferably from about 0.02 to about 8% by weight, more preferably from about 0.05 to about 6% by weight, most preferably from about 0.1 to about 4% by weight, based on the total weight of the cosmetic product, in the cosmetic product, and/or the organic silicon compound of the formula (II), in particular the 3-(triethoxysilyl)-N-[3-(triethoxysilyl)propyl]-1-propanamine, is present in an amount of from about 0.01 to about 10% by weight, preferably from about 0.02 to about 9% by weight, more preferably from about 0.05 to about 8% by weight, most preferably from about 0.1 to about 7%, by weight, based on the total weight of the cosmetic composition, in the cosmetic composition.

It was found that particularly stable and uniform films could be obtained on the keratinous material even when the agent contained two structurally different organic silicon compounds.

In a preferred embodiment, an agent exemplified comprises at least one organic silicone compound of formula (I) and at least one organic silicone compound of formula (IV).

In an explicitly very particularly preferred embodiment, an agent exemplified contains at least one organic silicone compound of formula (I) selected from the group consisting of (3-aminopropyl)triethoxysilane and (3-aminopropyl)trimethoxysilane, and additionally containing at least one organic silicone compound of formula (IV) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an agent is exemplified in that the agent-contains, based on the total weight of the agent-:

from about 0.5 to about 5% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and

from about 3.2 to about 10% by weight of at least one second organic silicon compound selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octyldecyltrimethoxysilane and octyldecyltriethoxysilane.

Even the addition of small amounts of water leads to hydrolysis in organic silicon compounds with at least one hydrolyzable group. The hydrolysis products and/or organic silicon compounds having at least one hydroxy group may react with each other in a condensation reaction. For this reason, both the organosilicon compounds having at least one hydrolyzable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

In the context of the present disclosure, figures in wt.-% are—unless otherwise stated—always based on the total weight of the cosmetic product.

As a second ingredient, the cosmetic composition for treating a keratinous material contains a sulfonated and/or sulfated fatty acid ester. In the course of the work leading to the present disclosure, it has been found that, in order to achieve a particularly good care effect, it is particularly advantageous if the organic silicon compounds, for example (3-aminopropyl)triethoxysilane, i.e. an aminopropyltriethoxysilane (AMEO), or for example 3-(triethoxysilyl)-N-[3-(triethoxysilyl)propyl]-1-propanamine, i.e. a bis(triethoxysilylpropyl)amine, is combined with a sulfated and/or sulfonated fatty acid ester, preferably a sulfated and/or sulfonated fatty acid triglyceride.

It has been found that the combination of sulfated and/or sulfonated fatty acid triglyceride and the aminopropyltriethoxysilane (AMEO) and/or the bis(triethoxysilylpropyl)amine is particularly powerful in terms of maintenance. The hair surface is hydrophobized, reducing frizz. The hair becomes softer and even with a high chemical stress, the hair can still be shaped better.

According to a preferred embodiment, one or more sulfated/sulfonated fatty acid triglycerides are included in the cosmetic composition, which together form a total amount. According to this preferred embodiment, the total amount of sulfated/sulfonated fatty acid triglyceride is about 0.1 to about 30% by weight, preferably from about 0.5 to about 20% by weight, more preferably from about 1 to about 10% by weight, based on the total weight of the cosmetic product.

According to a preferred embodiment, sulfated and/or sulfonated fatty acid triglycerides in the cosmetic composition comprise fatty acid triglycerides whose fatty acid groups are linear or branched, saturated or unsaturated, and/or hydroxy-functionalized, wherein the fatty acid groups have 8 to 30, preferably 10 to 26, more preferably 12-24 C atoms. Sulfonation or sulfation may have occurred at the unsaturated binding sites and/or at the hydroxy functions.

Particularly preferred sulfated/sulfonated fatty acid triglycerides are sulfated vegetable oils, preferably sulfated canola oil (INCI: Sulfated Rapeseed Oil), sulfated sunflower oil (INCI: Sulfated Sunflower Seed Oil), sulfated coconut oil (INCI: Sulfated Coconut Oil), sulfated castor oil (INCI: Sulfated Castor Oil), sulfated meadow foam oil (also known as marsh flower oil), sulfated olive oil (INCI: Sulfated Olive Oil), sulfated soybean oil, and especially sulfated coconut oil (INCI: Sulfated Coconut Oil), sulfated canola oil (INCI: Sulfated Rapeseed Oil) and/or sulfated castor oil (INCI: Sulfated Castor Oil).

Most preferably, the cosmetic composition contains sulfated castor oil corresponding to the following formula:

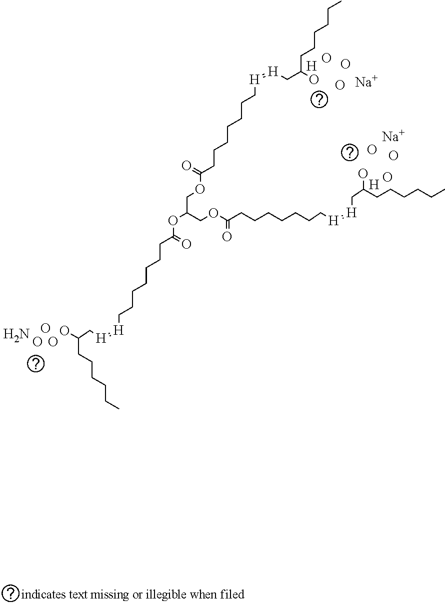

In particular, the features for treating a keratinous material may comprise a feature for cleaning a keratinous material, a feature for maintaining a keratinous material, a feature for maintaining and cleaning a keratinous material, and/or a feature for temporarily reshaping a keratinous material.

In the following, further ingredients of the hair treatment products are described, which may be contained in the products in addition to the previously described mandatory ingredients.

It may be preferred that the agent for treating a keratinous material further comprises from about 0.001 to about 20% by weight of at least one quaternary compound. This applies to agents for the care of a keratinous material and agents for the care and cleaning of a keratinous material.

It is preferred that the at least one quaternary compound is selected from at least one of the groups consisting of:

i) of the monoalkylquats and/or
ii) the esterquats and/or
iii) of the quaternary imidazolines of formula (Tkat2),

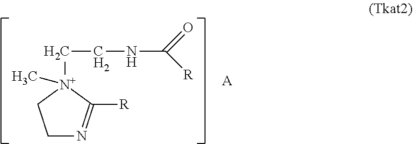

in which the radicals R independently of one another each represent a saturated or unsaturated, linear, or branched hydrocarbon radical having a chain length of 8 to 30 carbon atoms and A represents a physiologically tolerated anion, and/or
iv) the amidoamines and/or cationized amidoamines and/or
v) Poly(methacryloyloxyethyltrimethylammonium compounds) and/or;
vi) quaternized cellulose derivatives, polyquaternium 10, polyquaternium-24, polyquaternium-27, polyquaternium-67, polyquaternium-72, and/or
vii) cationic alkyl polyglycosides and/or
viii) cationized honey and/or
ix) cationic guar derivatives and/or
x) Chitosan and/or
xi) polymeric dimethyldiallylammonium salts and copolymers thereof with esters and amides of acrylic acid and methacrylic acid, in particular polyquaternium-7 and/or
xii) Copolymers of vinylpyrrolidone with quaternized derivatives of dialkylaminoalkyl acrylate and methacrylate, especially polyquaternium-11 and/or
xiii) vinylpyrrolidone-vinylimidazolium methochloride copolymers, in particular polyquaternium-16 and/or
xiv) quaternized polyvinyl alcohol and/or

### xv) Polyquaternium-74,

and mixtures thereof.

It is particularly preferred that the hair treatment composition contains a cationic hompolymer falling under the INCI designation polyquaternium-37 as quaternary compounds.

It may be preferred that the agent for treating a keratinous material further comprises a firming compound, preferably selected from the group consisting of waxes, synthetic polymers, and mixtures thereof.

To meet the different requirements for agents for the treatment of a keratinous material in the form of an agent for the temporary reshaping of a keratinous material (=styling agent), many synthetic polymers have already been developed as strengthening compounds, which can be used in the agent for the treatment of a keratinous material. Alternatively, or complementarily, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic, and amphoteric strengthening polymers.

Suitable synthetic polymers include, for example, polymers with the following INCI designations: Acrylamides/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Ammonium Methacrylate Copolymer, Acrylates/t-Butyl Acrylamide Copolymer, Acrylates Copolymer, Acrylates/C₁-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopic Acid ntyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetone Acrylamide Copolymer, Ammonium-Acrylates/VA/Acrylates Copolymer, AMPD-Acrylates/Acrylamides Copolymer, AMPD-Acrylates/Acrylamides/Allyl Methacrylate Copolymer, AMP-Acrylates/C₁-18 Alkyl Acrylates/C₁-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-hydroxypropyl Dimethicone Acrylate Copolymer, Isopropyl Ester of PVM/MA Acrylate Crosspolymer, Lauryl Methacrylate/Glycol Dimethacrylate Crosspolymer, MEA-Sulphite, Methacrylic Acid/Sodium Acrylamidomethyl Propane Sulphonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophen-2, Polyquaternium-2 Polyquaternium-4, Polyquaternium-5, Pol yquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, 18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polquaternium-56 9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 polyglyce ryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylbiloxylsilicon VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Also, homopolyacrylic acid (INCI: Carbomer), which is commercially available under the name Carbopol® in various forms, is suitable as a firming compound.

Preferably, the firming compound comprises a vinylpyrrolidone-containing polymer. Particularly preferably, the firming compound comprises a polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another preferred solidifying compound is octylacrylamide/acrylates/butylaminoethyl methacrylate copolymer (INCI), which is marketed under the name “Amphomer®” by Akzo Nobel.

Accordingly, it is particularly preferred that the firming compound comprises a synthetic polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

According to further preferred embodiments, the cosmetic composition comprises at least one cationic surfactant of formula (V),

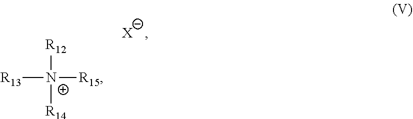

wherein
R₁₂, R₁₃, R₁₄ independently represent a C1-C6 alkyl group, a C2-C6 alkenyl group or a C2-C6 hydroxyalkyl group,
R₁₅ is a C8-C28 alkyl group, preferably a C10-C22 alkyl group, and
X— represents a physiologically compatible anion,
and/or the cosmetic composition comprises at least one cationic surfactant of the formula (VI),

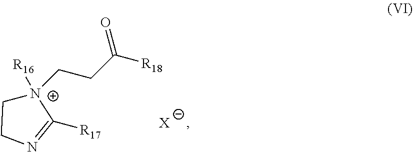

wherein
R₁₆ represents a C1-C6 alkyl group
R₁₇, R₁₈ independently represent a C7-C27 alkyl group, preferably a C10-C22 alkyl group, and
X— represents a physiologically compatible anion,
and/or the cosmetic composition comprises at least one cationic surfactant of the formula (VII),

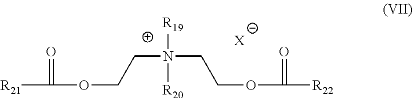

wherein
R₁₉, R₂₀ independently represent a C1-C6 alkyl group or a C2-C6 hydroxyalkyl group,
R₂₁, R₂₂ independently represent a C7-C27 alkyl group, preferably a C10-C22 alkyl group, and
X— represents a physiologically compatible anion,
and/or the cosmetic composition comprises at least one cationic surfactant of the formula (VIII),

NR₂₃R₂₄R₂₅ (VIII)

wherein
R₂₃, R₂₄ independently represent a C1-C6 alkyl group, a C2-C6 alkenyl group or a C2-C6 hydroxyalkyl group, and
R₂₅ represents a C8-C28 alkyl group, preferably a C10-C22 alkyl group,

The cationic surfactants of formula (VIII) are amine derivatives, so-called pseudoquats. The organic radicals R₂₃, R₂₄ and R₂₅ are directly bonded to the nitrogen atom. In the acidic pH range, these are cationized, i.e., the nitrogen atom is then protonated. The physiologically compatible counterions are suitable as counterions. Steamidopropyl dimethylamine is particularly preferred among the cationic surfactants of formula (VIII).

According to a preferred embodiment, the amount of cationic surfactant is about 0.1 to about 30% by weight, preferably from about 0.5 to about 20% by weight, more preferably from about 1 to about 10% by weight, based on the total weight of the cosmetic composition.

According to a preferred embodiment, the cationic surfactant comprises a hydrophobic head group with a cationic charge and one or two hydrophobic end portions, wherein the hydrophobic end portion(s) represent straight-chain or branched, saturated or mono- or polyunsaturated alkyl groups, preferably having a chain length of C6 to C30, more preferably C8 to C26, particularly preferably C10 to C22. According to another preferred embodiment, the cationic surfactant has an ester function, an ether function, a ketone function, an alcohol function, or an amide function.

According to a preferred embodiment, the cosmetic composition further comprises a nonionic surfactant. This preferably comprises a nonionic surfactant selected from the group consisting of:

Alkylglucamide comprising a saturated or unsaturated, branched, or unbranched C₆ to C₂₂, preferably C₁₀ to C₁₈, more preferably C₁₂ to C₁₆ alkyl group,

Alkyl fructoside comprising a saturated or unsaturated, branched, or unbranched C₆ to C₂₂, preferably C₁₀ to C₁₈, more preferably C₁₂ to C₁₆ alkyl group,

An alkyl glucoside comprising a saturated or unsaturated, branched, or unbranched C₆ to C₂₂, preferably C₁₀ to C₁₈, more preferably C₁₂ to C₁₆ alkyl group, and

Alkyl alcohol alkoxylate of the formula R₁₀(OR₁₁)ₘOH, in which R₁₀ represents a linear or branched C₆-C₂₂, preferably C₁₀-C₁₈, more preferably C₁₂-C₁₆ alkyl group, R₁₁ represents a C₂-C₄, preferably a C₂ alkyl group, and m represents 1 to 10, preferably 2 to 6, more preferably 2 to 6,

According to preferred embodiments, one or more anionic surfactants are included in the cosmetic composition, which is preferably selected from the group consisting of:

straight-chain or branched, saturated or mono- or polyunsaturated alkyl sulfonates containing 8 to 24, preferably 12 to 22, more preferably 16 to 18 carbon atoms,

linear alpha-olefin sulfonates with 8 to 24, preferably 12 to 22, more preferably 16 to 18 C atoms,

Alkyl sulfates and alkyl polyglycol ether sulfates of the formula R₉—O—(CH₂—CH₂O)ₙ—SO₃X, in which R₉ is preferably a straight-chain or branched, saturated or mono- or polyunsaturated alkyl or alkenyl radical having 8 to 24, preferably 12 to 22, more preferably 16 to 18 carbon atoms, n is 0 or 1 to 12, more preferably 2 to 4, and X is an alkali metal or alkaline earth metal ion or protonated triethanolamine or the ammonium ion,

straight-chain or branched, saturated or mono- or polyunsaturated alkyl carboxylic acids containing 8 to 24, preferably 12 to 22, more preferably 16 to 18 carbon atoms,

straight-chain or branched, saturated or mono- or polyunsaturated alkyl phosphates containing 8 to 24, preferably 12 to 22, more preferably 16 to 18 carbon atoms,

Alkyl isethionate whose alkyl group is selected from a branched or unbranched C₆ to C₂₂, preferably C₁₀ to C₁₈, more preferably C₁₂ to C₁₆ alkyl group, in particular sodium cocoyl isethionate,

Alkyl glycoside carboxylic acids whose alkyl group is selected from a branched or unbranched C₆ to C₂₂, preferably C₁₀ to C₁₈, more preferably C₁₂ to C₁₆ alkyl group,

Alkyl sulfosuccinates, the two alkyl groups of which are selected from identical or different, branched, or unbranched C₂ to C₁₂, preferably C₄ to C₁₀, more preferably C₆ to C₈ alkyl groups,

Alkyl taurates, the alkyl group of which is selected from a branched or unbranched C₆ to C₂₂, preferably C₁₀ to C₁₈, more preferably C₁₂ to C₁₆ alkyl group,

Alkyl sarcosinates whose alkyl group is selected from a branched or unbranched C₆ to C₂₂, preferably C₁₀ to C₁₈, more preferably C₁₂ to C₁₆ alkyl group,

Sulfonates of unsaturated fatty acids with 8 to 24, preferably 12 to 22, more preferably 16 to 18 C atoms and 1 to 6 double bonds,

wherein the counterion of the anionic surfactant is an alkali or alkaline earth metal ion or a protonated triethanolamine or the ammonium ion.

Particularly preferred anionic surfactants are straight-chain or branched alkyl ether sulfates containing an alkyl radical with 8 to 18 and with 10 to 16 carbon atoms and 1 to 6 and 2 to 4 ethylene oxide units. Very preferably, the surfactant mixture of anionic and amphoteric/zwitterionic surfactants contains sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO⁻ or —SO₃⁻ group in the molecule. Amphoteric/zwitterionic surfactants also include surface-active compounds which, in addition to a C₈-C₂₄ alkyl or acyl group, contain at least one free amino group and at least one —COOH— or —SO₃H— group and can form internal salts.

According to a preferred embodiment, the amphoteric surfactants in the cosmetic composition are selected from the group consisting of:

Alkyl betaine comprising at least one saturated or unsaturated, branched, or unbranched C₆ to C₂₂, preferably C₁₀ to C₁₈, more preferably C₁₂ to C₁₆ alkyl group,

Alkyl amphodiacetate or alkyl amphodiacetate comprising a saturated or unsaturated, branched, or unbranched C₆ to C₂₂, preferably C₁₀ to C₁₈, more preferably C₁₂ to C₁₆ alkyl group, with an alkali or alkaline earth metal counterion, and

Alkylamidopropyl betaine comprising at least one saturated or unsaturated, branched, or unbranched C₆ to C₂₂, preferably C₁₀ to C₁₈, more preferably C₁₂ to C₁₆ alkyl group.

Particularly suitable amphoteric/zwitterionic surfactants include those known under the INCI designation cocamidopropyl betaine and disodium cocoamphodiacetate.

According to a preferred embodiment, the nonionic surfactant is selected from the group consisting of:

Alkylglucamide comprising a saturated or unsaturated, branched, or unbranched C₆ to C₂₂, preferably C₁₀ to C₁₈, more preferably C₁₂ to C₁₆ alkyl group,

Alkyl fructoside comprising a saturated or unsaturated, branched, or unbranched C₆ to C₂₂, preferably C₁₀ to C₁₈, more preferably C₁₂ to C₁₆ alkyl group,

An alkyl glucoside comprising a saturated or unsaturated, branched, or unbranched C₆ to C₂₂, preferably C₁₀ to C₁₈, more preferably C₁₂ to C₁₆ alkyl group,

Alkyl alcohol alkoxylate of the formula R₁₀(OR₁₁)ₘOH, in which R₁₀ represents a linear or branched C₆-C₂₂, preferably C₁₀-C₁₈, more preferably C₁₂-C₁₆ alkyl group, R₁₁ represents a C₂-C₄, preferably a C₂ alkyl group, and m represents 1 to 10, preferably 2 to 6, more preferably 2 to 6, and

Alkyl esters of the formula R₁₂COOR₁₃, in which R₁₂ represents a linear or branched C₆ to C₂₂, preferably C₁₀ to C₁₈, more preferably C₁₂ to C₁₆ alkyl group, R₁₃ represents a C₁ to C₄, preferably a C₂ alkyl group.

According to a preferred embodiment, the cosmetic composition contains two structurally different surfactants. It is particularly preferred that the cosmetic agent contains two structurally different surfactants from one another, preferably the cosmetic agent contains two structurally different cationic surfactants from one another, or the cosmetic agent contains a cationic surfactant and a nonionic surfactant.

The cosmetic composition may contain, in addition or as an alternative to a synthetic polymer, at least one natural or synthetic wax having a melting point above 37° C. as a firming compound.

Natural or synthetic waxes can be solid kerosenes or isoparaffins, vegetable waxes such as candelilla wax, camauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Furthermore, in addition to the mandatory components, the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate are suitable in the cosmetic products.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. Thus, several waxes can also be used. Furthermore, several wax mixtures, possibly mixed with other additives, are also commercially available. The products sold under the designations “Special Wax 7686 OE” (a mixture of cetyl palmitate, beeswax, microcrystalline wax and polyethylene with a melting range of 73-75° C.; manufacturer: Kahl & Co), Polywax® GP 200 (a mixture of stearyl alcohol and polyethylene glycol stearate with a melting point of 47-51° C.; manufacturer: Croda) and “Softceresin® FL 400” (a vaseline/vaseline oil/wax mixture with a melting point of 50-54° C.; manufacturer: Parafluid Mineral Oil Company) are examples of mixtures that can be used.

Preferably, the wax is selected from carnauba wax (INCI: Copernicia Cerifera Cera) Bienenwachs (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and petrolatum.

The wax or wax components should be solid at 25° C. and should melt in the range >37° C.

The composition for treating a keratinous material preferably contains the firming compound in a total amount of about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic polymers, anionic polymers, (further) cationic polymers, waxes, protein hydrolysates, amino acids, oligopetides, vitamins, provitamins, vitamin precursors, betaines, bioquinones, purine (derivatives), care substances, plant extracts, silicones, ester oils, UV light filters, structuring agents, thickening agents, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

In preferred embodiments 1 to 337, the organic silicon compounds of the following Table are combined with a sulfated vegetable oil of the following Table in a cosmetic composition as contemplated herein.

| | Silane compound | other ingredient |
|---|---|---|
| 1 | -(3-Aminopropyl)trimethoxysilane | Sulfated Castor Oil (INCI) |
| 2 | (3-Aminopropyl)triethoxysilan | Sulfated Castor Oil (INCI) |
| 3 | -(2-Aminoethyl)trimethoxysilane | Sulfated Castor Oil (INCI) |
| 4 | -(2-Aminoethyl)triethoxysilane | Sulfated Castor Oil (INCI) |
| 5 | -(3-Dimethylaminopropyl)trimethoxysilane | Sulfated Castor Oil (INCI) |
| 6 | (3-Dimethylaminopropyl)triethoxysilane | Sulfated Castor Oil (INCI) |
| 7 | (2-Dimethylaminoethyl)trimethoxysilane | Sulfated Castor Oil (INCI) |
| 8 | (2-Dimethylaminoethyl)triethoxysilane | Sulfated Castor Oil (INCI) |
| 9 | 3-(trimethoxys ilyl)-N-[3- | Sulfated Castor Oil (INCI) |
| | (trimethoxysilyl)propyl]-1-propanamine | |
| 10 | 3-(Triethoxysilyl)-N-[3-(triethoxysilyl)propyl]-1- | Sulfated Castor Oil (INCI) |
| | propanamine | |
| 11 | N-methyl-3-(trimethoxysilyl)-N-[3- | Sulfated Castor Oil (INCI) |
| | (trimethoxysilyl)propyl]-1-propanamine | |
| 12 | N-Methyl-3-(triethoxysilyl)-N-[3- | Sulfated Castor Oil (INCI) |
| | (triethoxysilyl)propyl]-1-propanamine | |
| 13 | 2-[Bis[3-(trimethoxysilyl)propyl]amino]-ethanol | Sulfated Castor Oil (INCI) |
| 14 | 2-[bis [3-(triethoxysilyl)propyl]amino]ethanol | Sulfated Castor Oil (INCI) |
| 15 | 3-(Trimethoxysilyl)-N,N-bis[3- | Sulfated Castor Oil (INCI) |
| | (trimethoxysilyl)propyl]-1-propanamine | |
| 16 | 3-(Triethoxysilyl)-N,N-bis[3- | Sulfated Castor Oil (INCI) |
| | (triethoxysilyl)propyl-1-propanamine | |
| 17 | N1,N1-Bis[3-(trimethoxysilyl)propyl]-1,2- | Sulfated Castor Oil (INCI) |
| | ethanediamine | |
| 18 | N1,N1-Bis[3-(triethoxysilyl)propyl]-1,2- | Sulfated Castor Oil (INCI) |
| | ethanediamine | |
| 19 | N,N-Bis[3-(trimethoxysilyl)propyl]-2-propen-1- | Sulfated Castor Oil (INCI) |
| | amine | |
| 20 | N,N-Bis[3-(triethoxysilyl)propyl]-2-propen-1- | Sulfated Castor Oil (INCI) |
| | amine | |
| 21 | Methyltrimethoxysilane | Sulfated Castor Oil (INCI) |
| 22 | Methyltriethoxysilane | Sulfated Castor Oil (INCI) |
| 23 | Ethyltrimethoxysilane | Sulfated Castor Oil (INCI) |
| 24 | Ethyltriethoxysilane | Sulfated Castor Oil (INCI) |
| 25 | Octyltrimethoxysilane | Sulfated Castor Oil (INCI) |
| 26 | Octyltriethoxysilane | Sulfated Castor Oil (INCI) |
| 27 | Dodecyltrimethoxysilane | Sulfated Castor Oil (INCI) |
| 28 | Dodecyltriethoxysilane | Sulfated Castor Oil (INCI) |
| 29 | -(3-Aminopropyl)trimethoxysilane | Sulfated Rapeseed Oil (INCI) |
| 30 | (3-Aminopropyl)triethoxysilan | Sulfated Rapeseed Oil (INCI) |
| 31 | -(2-Aminoethyl)trimethoxysilane | Sulfated Rapeseed Oil (INCI) |
| 32 | -(2-Aminoethyl)triethoxysilane | Sulfated Rapeseed Oil (INCI) |
| 33 | -(3-Dimethylaminopropyl)trimethoxysilane | Sulfated Rapeseed Oil (INCI) |
| 34 | (3-Dimethylaminopropyl)triethoxysilane | Sulfated Rapeseed Oil (INCI) |
| 35 | (2-Dimethylaminoethyl)trimethoxysilane | Sulfated Rapeseed Oil (INCI) |
| 36 | (2-Dimethylaminoethyl)triethoxysilane | Sulfated Rapeseed Oil (INCI) |
| 37 | 3-(trimethoxysilyl)-N-[3- | Sulfated Rapeseed Oil (INCI) |
| | (trimethoxysilyl)propyl]-1-propanamine | |
| 38 | 3-(Triethoxysilyl)-N-[3-(triethoxysilyl)propyl]-1- | Sulfated Rapeseed Oil (INCI) |
| | propanamine | |
| 39 | N-methyl-3-(trimethoxysilyl)-N-[3- | Sulfated Rapeseed Oil (INCI) |
| | (trimethoxysilyl)propyl]-1-propanamine | |
| 40 | N-Methyl-3-(triethoxysilyl)-N-[3- | Sulfated Rapeseed Oil (INCI) |
| | (triethoxysilyl)propyl]-1-propanamine | |
| 41 | 2-[Bis[3-(trimethoxysilyl)propyl]amino]-ethanol | Sulfated Rapeseed Oil (INCI) |
| 42 | 2-[bis[3-(triethoxysilyl)propyl]amino]ethanol | Sulfated Rapeseed Oil (INCI) |
| 43 | 3-(Trimethoxysilyl)-N,N-bis[3- | Sulfated Rapeseed Oil (INCI) |
| | (trimethoxysilyl)propyl]-1-propanamine | |
| 44 | 3-(Triethoxysilyl)-N,N-bis[3- | Sulfated Rapeseed Oil (INCI) |
| | (triethoxysilyl)propyl]-1-propanamine | |
| 45 | N1,N1-Bis[3-(trimethoxysilyl)propyl]-1,2- | |
| | ethanediamine | |
| 46 | N1,N1-Bis[3-(triethoxysilyl)propyl]-1,2- | Sulfated Rapeseed Oil (INCI) |
| | ethanediamine | |
| 47 | N,N-Bis[3-(trimethoxysilyl)propyl]-2-propen-1- | Sulfated Rapeseed Oil (INCI) |
| | amine | |
| 48 | N,N-Bis[3-(triethoxysilyl)propyl]-2-propen-1- | Sulfated Rapeseed Oil (INCI) |
| | amine | |
| 49 | Methyltrimethoxysilane | Sulfated Rapeseed Oil (INCI) |
| 50 | Methyltriethoxysilane | Sulfated Rapeseed Oil (INCI) |
| 51 | Ethyltrimethoxysilane | Sulfated Rapeseed Oil (INCI) |
| 52 | Ethyltriethoxysilane | Sulfated Rapeseed Oil (INCI) |
| 53 | Octyltrimethoxysilane | Sulfated Rapeseed Oil (INCI) |
| 54 | Octyltriethoxysilane | Sulfated Rapeseed Oil (INCI) |
| 55 | Dodecyltrimethoxysilane | Sulfated Rapeseed Oil (INCI) |
| 56 | Dodecyltriethoxysilane | Sulfated Rapeseed Oil (INCI) |
| 57 | -(3-Aminopropyl)trimethoxysilane | Sulfated Coconut Oil (INCI) |
| 58 | (3-Aminopropyl)triethoxysilan | Sulfated Coconut Oil (INCI) |
| 59 | -(2-Aminoethyl)trimethoxysilane | Sulfated Coconut Oil (INCI) |
| 60 | -(2-Aminoethyl)triethoxysilane | Sulfated Coconut Oil (INCI) |
| 61 | -(3-Dimethylaminopropyl)trimethoxysilane | Sulfated Coconut Oil (INCI) |
| 62 | (3-Dimethylaminopropyl)triethoxysilane | Sulfated Coconut Oil (INCI) |
| 63 | (2-Dimethylaminoethyl)trimethoxysilane | Sulfated Coconut Oil (INCI) |
| 64 | (2-Dimethylaminoethyl)triethoxysilane | Sulfated Coconut Oil (INCI) |
| 65 | 3-(trimethoxysilyl)-N-[3- | Sulfated Coconut Oil (INCI) |
| | (trimethoxysilyl)propyl]-1-propanamine | |
| 66 | 3-(Triethoxysilyl)-N-[3-(triethoxysilyl)propyl]-1- | Sulfated Coconut Oil (INCI) |
| | propanamine | |
| 67 | N-methyl-3-(trimethoxysilyl)-N-[3- | Sulfated Coconut Oil (INCI) |
| | (trimethoxysilyl)propyl]-1-propanamine | |
| 68 | N-Methyl-3-(triethoxysilyl)-N-[3- | Sulfated Coconut Oil (INCI) |
| | (triethoxysilyl)propyl]-1-propanamine | |
| 69 | 2-[Bis[3-(trimethoxysilyl)propyl]amino]-ethanol | Sulfated Coconut Oil (INCI) |
| 70 | 2-[bis[3-(triethoxysilyl)propyl]amino]ethanol | Sulfated Coconut Oil (INCI) |
| 71 | 3-(Trimethoxysilyl)-N,N-bis[3- | Sulfated Coconut Oil (INCI) |
| | (trimethoxysilyl)propyl]-1-propanamine | |
| 72 | 3-(Triethoxysilyl)-N,N-bis[3- | Sulfated Coconut Oil (INCI) |
| | (triethoxysilyl)propyl]-1-propanamine | |
| 73 | N1,N1-Bis[3-(trimethoxysilyl)propyl]-1,2- | Sulfated Coconut Oil (INCI) |
| | ethanediamine | |
| 74 | N1,N1-Bis[3-(triethoxysilyl)propyl]-1,2- | Sulfated Coconut Oil (INCI) |
| | ethanediamine | |
| 75 | N,N-Bis[3-(trimethoxysilyl)propyl]-2-propen-1- | Sulfated Coconut Oil (INCI) |
| | amine | |
| 76 | N,N-Bis[3-(triethoxysilyl)propyl]-2-propen-1- | Sulfated Coconut Oil (INCI) |
| | amine | |
| 77 | Methyltrimethoxysilane | Sulfated Coconut Oil (INCI) |
| 78 | Methyltriethoxysilane | Sulfated Coconut Oil (INCI) |
| 79 | Ethyltrimethoxysilane | Sulfated Coconut Oil (INCI) |
| 80 | Ethyltriethoxysilane | Sulfated Coconut Oil (INCI) |
| 81 | Octyltrimethoxysilane | Sulfated Coconut Oil (INCI) |
| 82 | Octyltriethoxysilane | Sulfated Coconut Oil (INCI) |
| 83 | Dodecyltrimethoxysilane | Sulfated Coconut Oil (INCI) |
| 84 | Dodecyltriethoxysilane | Sulfated Coconut Oil (INCI) |
| 85 | -(3-Aminopropyl)trimethoxysilane | sulfated soybean oil |
| 86 | (3-Aminopropyl)triethoxysilan | sulfated soybean oil |
| 87 | -(2-Aminoethyl)trimethoxysilane | sulfated soybean oil |
| 88 | -(2-Aminoethyl)triethoxysilane | sulfated soybean oil |
| 89 | -(3-Dimethylaminopropyl)trimethoxysilane | sulfated soybean oil |
| 90 | (3-Dimethylaminopropyl)triethoxysilane | sulfated soybean oil |
| 91 | (2-Dimethylaminoethyl)trimethoxysilane | sulfated soybean oil |
| 92 | (2-Dimethylaminoethyl)triethoxysilane | sulfated soybean oil |
| 93 | 3-(trimethoxysilyl)-N-[3- | sulfated soybean oil |
| | (trimethoxysilyl)propyl]-1-propanamine | |
| 94 | 3-(Triethoxysilyl)-N-[3-(triethoxysilyl)propyl]-1- | sulfated soybean oil |
| | propanamine | |
| 95 | N-methyl-3-(trimethoxysilyl)-N-[3- | sulfated soybean oil |
| | (trimethoxysilyl)propyl]-1-propanamine | |
| 96 | N-Methyl-3-(triethoxysilyl)-N-[3- | sulfated soybean oil |
| | (triethoxysilyl)propyl]-1-propanamine | |
| 97 | 2-[Bis[3-(trimethoxysilyl)propyl]amino]-ethanol | sulfated soybean oil |
| 98 | 2-[bis[3-(triethoxysilyl)propyl]amino]ethanol | |
| 99 | 3-(Trimethoxysilyl)-N,N-bis[3- | sulfated soybean oil |
| | (trimethoxysilyl)propyl]-1-propanamine | |
| 100 | 3-(Triethoxysilyl)-N,N-bis[3- | sulfated soybean oil |
| | (triethoxysilyl)propyl]-1-propanamine | |
| 101 | N1,N1-Bis[3-(trimethoxysilyl)propyl]-1,2- | sulfated soybean oil |
| | ethanediamine | |
| 102 | N1,N1-Bis[3-(triethoxysilyl)propyl]-1,2- | sulfated soybean oil |
| | ethanediamine | |
| 103 | N,N-Bis[3-(trimethoxysilyl)propyl]-2-propen-1- | sulfated soybean oil |
| | amine | |
| 104 | N,N-Bis[3-(triethoxysilyl)propyl]-2-propen-1- | sulfated soybean oil |
| | amine | |
| 105 | Methyltrimethoxysilane | sulfated soybean oil |
| 106 | Methyltriethoxysilane | sulfated soybean oil |
| 107 | Ethyltrimethoxysilane | sulfated soybean oil |
| 108 | Ethyltriethoxysilane | sulfated soybean oil |
| 109 | Octyltrimethoxysilane | sulfated soybean oil |
| 110 | Octyltriethoxysilane | sulfated soybean oil |
| 111 | Dodecyltrimethoxysilane | sulfated soybean oil |
| 112 | Dodecyltriethoxysilane | sulfated soybean oil |
| 113 | -(3-Aminopropyl)trimethoxysilane | Sulfated Olive Oil (INCI) |
| 114 | (3-Aminopropyl)triethoxysilan | Sulfated Olive Oil (INCI) |
| 115 | -(2-Aminoethyl)trimethoxysilane | Sulfated Olive Oil (INCI) |
| 116 | -(2-Aminoethyl)triethoxysilane | Sulfated Olive Oil (INCI) |
| 117 | -(3-Dimethylaminopropyl)trimethoxysilane | Sulfated Olive Oil (INCI) |
| 118 | (3-Dimethylaminopropyl)triethoxysilane | Sulfated Olive Oil (INCI) |
| 119 | (2-Dimethylaminoethyl)trimethoxysilane | Sulfated Olive Oil (INCI) |
| 120 | (2-Dimethylaminoethyl)triethoxysilane | Sulfated Olive Oil (INCI) |
| 121 | 3-(trimethoxysilyl)-N-[3- | Sulfated Olive Oil (INCI) |
| | (trimethoxysilyl)propyl]-1-propanamine | |
| 122 | 3-(Triethoxysilyl)-N-[3-(triethoxysilyl)propyl]-1- | Sulfated Olive Oil (INCI) |
| | propanamine | |
| 123 | N-methyl-3-(trimethoxysilyl)-N-[3- | Sulfated Olive Oil (INCI) |
| | (trimethoxysilyl)propyl]-1-propanamine | |
| 124 | N-Methyl-3-(triethoxysilyl)-N-[3- | Sulfated Olive Oil (INCI) |
| | (triethoxysilyl)propyl]-1-propanamine | |
| 125 | 2-[Bis[3-(trimethoxysilyl)propyl]amino]-ethanol | Sulfated Olive Oil (INCI) |
| 126 | 2-[bis[3-(triethoxysilyl)propyl]amino]ethanol | Sulfated Olive Oil (INCI) |
| 127 | 3-(Trimethoxysilyl)-N,N-bis[3- | Sulfated Olive Oil (INCI) |
| | (trimethoxysilyl)propyl]-1-propanamine | |
| 128 | 3-(Triethoxysilyl)-N,N-bis[3- | Sulfated Olive Oil (INCI) |
| | (triethoxysilyl)propyl]-1-propanamine | |
| 129 | N1,N1-Bis[3-(trimethoxysilyl)propyl]-1,2- | Sulfated Olive Oil (INCI) |
| | ethanediamine | |
| 130 | N1,N1-Bis[3-(triethoxysilyl)propyl]-1,2- | Sulfated Olive Oil (INCI) |
| | ethanediamine | |
| 131 | N,N-Bis[3-(trimethoxysilyl)propyl]-2-propen-1- | Sulfated Olive Oil (INCI) |
| | amine | |
| 132 | N,N-Bis[3-(triethoxysilyl)propyl]-2-propen-1- | Sulfated Olive Oil (INCI) |
| | amine | |
| 133 | Methyltrimethoxysilane | Sulfated Olive Oil (INCI) |
| 134 | Methyltriethoxysilane | Sulfated Olive Oil (INCI) |
| 135 | Ethyltrimethoxysilane | Sulfated Olive Oil (INCI) |
| 136 | Ethyltriethoxysilane | Sulfated Olive Oil (INCI) |
| 137 | Octyltrimethoxysilane | Sulfated Olive Oil (INCI) |
| 138 | Octyltriethoxysilane | Sulfated Olive Oil (INCI) |
| 139 | Dodecyltrimethoxysilane | Sulfated Olive Oil (INCI) |
| 140 | Dodecyltriethoxysilane | Sulfated Olive Oil (INCI) |
| 141 | -(3-Aminopropyl)trimethoxysilane | sulfated meadow foam oil |
| 142 | (3-Aminopropyl)triethoxysilan | sulfated meadow foam oil |
| 143 | -(2-Aminoethyl)trimethoxysilane | sulfated meadow foam oil |
| 144 | -(2-Aminoethyl)triethoxysilane | sulfated meadow foam oil |
| 145 | -(3-Dimethylaminopropyl)trimethoxysilane | sulfated meadow foam oil |
| 146 | (3-Dimethylaminopropyl)triethoxysilane | sulfated meadow foam oil |
| 147 | (2-Dimethylaminoethyl)trimethoxysilane | sulfated meadow foam oil |
| 148 | (2-Dimethylaminoethyl)triethoxysilane | sulfated meadow foam oil |
| 149 | 3-(trimethoxysilyl)-N-[3- | sulfated meadow foam oil |
| | (trimethoxysilyl)propyl]-1-propanamine | |
| 150 | 3-(Triethoxysilyl)-N-[3-(triethoxysilyl)propyl]-1- | sulfated meadow foam oil |
| | propanamine | |
| 151 | N-methyl-3-(trimethoxysilyl)-N-[3- | sulfated meadow foam oil |
| | (trimethoxysilyl)propyl]-1-propanamine | |
| 152 | N-Methyl-3-(triethoxysilyl)-N-[3- | sulfated meadow foam oil |
| | (triethoxysilyl)propyl]-1-propanamine | |
| 153 | 2-[Bis[3-(trimethoxysilyl)propyl]amino]-ethanol | sulfated meadow foam oil |
| 154 | 2-[bis[3-(triethoxysilyl)propyl]amino]ethanol | sulfated meadow foam oil |
| 155 | 3-(Trimethoxysilyl)-N,N-bis[3- | sulfated meadow foam oil |
| | (trimethoxysilyl)propyl]-1-propanamine | |
| 156 | 3-(Triethoxysilyl)-N,N-bis[3- | sulfated meadow foam oil |
| | (triethoxysilyl)propyl]-1-propanamine | |
| 157 | N1,N1-Bis[3-(trimethoxysilyl)propyl]-1,2- | sulfated meadow foam oil |
| | ethanediamine | |
| 158 | N1,N1-Bis[3-(triethoxysilyl)propyl]-1,2- | sulfated meadow foam oil |
| | ethanediamine | |
| 159 | N,N-Bis[3-(trimethoxysilyl)propyl]-2-propen-1- | sulfated meadow foam oil |
| | amine | |
| 160 | N,N-Bis[3-(triethoxysilyl)propyl]-2-propen-1- | sulfated meadow foam oil |
| | amine | |
| 161 | Methyltrimethoxysilane | sulfated meadow foam oil |
| 162 | Methyltriethoxysilane | sulfated meadow foam oil |
| 163 | Ethyltrimethoxysilane | sulfated meadow foam oil |
| 164 | Ethyltriethoxysilane | sulfated meadow foam oil |
| 165 | Octyltrimethoxysilane | sulfated meadow foam oil |
| 166 | Octyltriethoxysilane | sulfated meadow foam oil |
| 167 | Dodecyltrimethoxysilane | sulfated meadow foam oil |
| 168 | Dodecyltriethoxysilane | sulfated meadow foam oil |
| 169 | -(3-Aminopropyl)trimethoxysilane | Sulfated Sunflower Seed Oil (INCI) |
| 170 | (3-Aminopropyl)triethoxysilan | Sulfated Sunflower Seed Oil (INCI) |
| 171 | -(2-Aminoethyl)trimethoxysilane | Sulfated Sunflower Seed Oil (INCI) |
| 172 | -(2-Aminoethyl)triethoxysilane | Sulfated Sunflower Seed Oil (INCI) |
| 173 | -(3-Dimethylaminopropyl)trimethoxysilane | Sulfated Sunflower Seed Oil (INCI) |
| 174 | (3-Dimethylaminopropyl)triethoxysilane | Sulfated Sunflower Seed Oil (INCI) |
| 175 | (2-Dimethylaminoethyl)trimethoxysilane | Sulfated Sunflower Seed Oil (INCI) |
| 176 | (2-Dimethylaminoethyl)triethoxysilane | Sulfated Sunflower Seed Oil (INCI) |
| 177 | 3-(trimethoxysilyl)-N-[3- | Sulfated Sunflower Seed Oil (INCI) |
| | (trimethoxysilyl)propyl]-1-propanamine | |
| 178 | 3-(Triethoxysilyl)-N-[3-(triethoxysilyl)propyl]-1- | Sulfated Sunflower Seed Oil (INCI) |
| | propanamine | |
| 179 | N-methyl-3-(trimethoxysilyl)-N-[3- | Sulfated Sunflower Seed Oil (INCI) |
| | (trimethoxysilyl)propyl]-1-propanamine | |
| 180 | N-Methyl-3-(triethoxysilyl)-N-[3- | Sulfated Sunflower Seed Oil (INCI) |
| | (triethoxysilyl)propyl]-1-propanamine | |
| 181 | 2-[Bis[3-(trimethoxysilyl)propyl]amino]-ethanol | Sulfated Sunflower Seed Oil (INCI) |
| 182 | 2-[bis[3-(triethoxysilyl)propyl]amino]ethanol | Sulfated Sunflower Seed Oil (INCI) |
| 183 | 3-(Trimethoxysilyl)-N,N-bis[3- | Sulfated Sunflower Seed Oil (INCI) |
| | (trimethoxysilyl)propyl]-1-propanamine | |
| 184 | 3-(Triethoxysilyl)-N,N-bis[3- | Sulfated Sunflower Seed Oil (INCI) |
| | (triethoxysilyl)propyl]-1-propanamine | |
| 185 | N1,N1-Bis[3-(trimethoxysilyl)propyl]-1,2- | Sulfated Sunflower Seed Oil (INCI) |
| | ethanediamine | |
| 186 | N1,N1-Bis[3-(triethoxysilyl)propyl]-1,2- | Sulfated Sunflower Seed Oil (INCI) |
| | ethanediamine | |
| 187 | N,N-Bis[3-(trimethoxysilyl)propyl]-2-propen-1- | Sulfated Sunflower Seed Oil (INCI) |
| | amine | |
| 188 | N,N-Bis[3-(triethoxysilyl)propyl]-2-propen-1- | Sulfated Sunflower Seed Oil (INCI) |
| | amine | |
| 189 | Methyltrimethoxysilane | Sulfated Sunflower Seed Oil (INCI) |
| 190 | Methyltriethoxysilane | Sulfated Sunflower Seed Oil (INCI) |
| 191 | Ethyltrimethoxysilane | Sulfated Sunflower Seed Oil (INCI) |
| 192 | Ethyltriethoxysilane | Sulfated Sunflower Seed Oil (INCI) |
| 193 | Octyltrimethoxysilane | Sulfated Sunflower Seed Oil (INCI) |
| 194 | Octyltriethoxysilane | Sulfated Sunflower Seed Oil (INCI) |
| 195 | Dodecyltrimethoxysilane | Sulfated Sunflower Seed Oil (INCI) |
| 196 | Dodecyltriethoxysilane | Sulfated Sunflower Seed Oil (INCI) |
| 197 | Propyltrimethoxysilane | Sulfated Castor Oil (INCI) |
| 198 | Propyltriethoxysilane | Sulfated Castor Oil (INCI) |
| 199 | Hexyltrimethoxysilane | Sulfated Castor Oil (INCI) |
| 200 | Hexyltriethoxysilane | Sulfated Castor Oil (INCI) |
| 201 | Octadecyltrimethoxysilane | Sulfated Castor Oil (INCI) |
| 202 | Octadecyltriethoxysilane | Sulfated Castor Oil (INCI) |
| 203 | Propyltrimethoxysilane | Sulfated Rapeseed Oil (INCI) |
| 204 | Propyltriethoxysilane | Sulfated Rapeseed Oil (INCI) |
| 205 | Hexyltrimethoxysilane | Sulfated Rapeseed Oil (INCI) |
| 206 | Hexyltriethoxysilane | Sulfated Rapeseed Oil (INCI) |
| 207 | Octadecyltrimethoxysilane | Sulfated Rapeseed Oil (INCI) |
| 208 | Octadecyltriethoxysilane | Sulfated Rapeseed Oil (INCI) |
| 209 | Propyltrimethoxysilane | Sulfated Olive Oil (INCI) |
| 210 | Propyltriethoxysilane | Sulfated Olive Oil (INCI) |
| 211 | Hexyltrimethoxysilane | Sulfated Olive Oil (INCI) |
| 212 | Hexyltriethoxysilane | Sulfated Olive Oil (INCI) |
| 213 | Octadecyltrimethoxysilane | Sulfated Olive Oil (INCI) |
| 214 | Octadecyltriethoxysilane | Sulfated Olive Oil (INCI) |
| 215 | Propyltrimethoxysilane | Sulfated Olive Oil (INCI) |
| 216 | Propyltriethoxysilane | Sulfated Sunflower Seed Oil (INCI) |
| 217 | Hexyltrimethoxysilane | Sulfated Sunflower Seed Oil (INCI) |
| 218 | Hexyltriethoxysilane | Sulfated Sunflower Seed Oil (INCI) |
| 219 | Octadecyltrimethoxysilane | Sulfated Sunflower Seed Oil (INCI) |
| 220 | Octadecyltriethoxysilane | Sulfated Sunflower Seed Oil (INCI) |
| 221 | Propyltrimethoxysilane | sulfated soybean oil |
| 222 | Propyltriethoxysilane | sulfated soybean oil |
| 223 | Hexyltrimethoxysilane | sulfated soybean oil |
| 224 | Hexyltriethoxysilane | sulfated soybean oil |
| 225 | Octadecyltrimethoxysilane | sulfated soybean oil |
| 226 | Octadecyltriethoxysilane | sulfated soybean oil |
| 227 | Propyltrimethoxysilane | sulfated meadow foam oil |
| 228 | Propyltriethoxysilane | sulfated meadow foam oil |
| 229 | Hexyltrimethoxysilane | sulfated meadow foam oil |
| 230 | Hexyltriethoxysilane | sulfated meadow foam oil |
| 231 | Octadecyltrimethoxysilane | sulfated meadow foam oil |
| 232 | Octadecyltriethoxysilane | sulfated meadow foam oil |
| 233 | Propyltrimethoxysilane | Sulfated Coconut Oil (INCI) |
| 234 | Propyltriethoxysilane | Sulfated Coconut Oil (INCI) |
| 235 | Hexyltrimethoxysilane | Sulfated Coconut Oil (INCI) |
| 236 | Hexyltriethoxysilane | Sulfated Coconut Oil (INCI) |
| 237 | Octadecyltrimethoxysilane | Sulfated Coconut Oil (INCI) |
| 238 | Octadecyltriethoxysilane | Sulfated Coconut Oil (INCI) |
| 239 | (3-Aminopropyl)triethoxysilane + | Sulfated Castor Oil (INCI) |
| | methyltrimethoxysilane | |
| 240 | (3-Aminopropyl)triethoxysilane + | Sulfated Castor Oil (INCI) |
| | methyltriethoxysilane | |
| 241 | (3-Aminopropyl)triethoxysilane + | Sulfated Castor Oil (INCI) |
| | ethyltrimethoxysilane | |
| 242 | (3-Aminopropyl)triethoxysilane + | Sulfated Castor Oil (INCI) |
| | ethyltriethoxysilane | |
| 243 | (3-Aminopropyl)triethoxysilane + | Sulfated Castor Oil (INCI) |
| | propyltrimethoxysilane | |
| 244 | (3-Aminopropyl)triethoxysilane + | Sulfated Castor Oil (INCI) |
| | propyltriethoxysilane | |
| 245 | (3-Aminopropyl)triethoxysilane + | Sulfated Castor Oil (INCI) |
| | hexyltrimethoxysilane | |
| 246 | (3-aminopropyl)triethoxysilane + | Sulfated Castor Oil (INCI) |
| | hexyltriethoxysilane | |
| 247 | (3-Aminopropyl)triethoxysilane + | Sulfated Castor Oil (INCI) |
| | octyltrimethoxysilane | |
| 248 | (3-Aminopropyl)triethoxysilane + | Sulfated Castor Oil (INCI) |
| | Octyltriethoxysilane | |
| 249 | (3-Aminopropyl)triethoxysilane + | Sulfated Castor Oil (INCI) |
| | dodecyltrimethoxysilane | |
| 250 | (3-Aminopropyl)triethoxysilane + | Sulfated Castor Oil (INCI) |
| | dodecyltriethoxysilane | |
| 251 | (3-aminopropyl)triethoxysilane + | Sulfated Castor Oil (INCI) |
| | octyldecyltrimethoxysilane | |
| 252 | (3-Aminopropyl)triethoxysilane + | Sulfated Castor Oil (INCI) |
| | Octyldecyltriethoxysilane | |
| 253 | (3-Aminopropyl)triethoxysilane + | Sulfated Rapeseed Oil (INCI) |
| | methyltrimethoxysilane | |
| 254 | (3-Aminopropyl)triethoxysilane + | Sulfated Rapeseed Oil (INCI) |
| | methyltriethoxysilane | |
| 255 | (3-Aminopropyl)triethoxysilane + | Sulfated Rapeseed Oil (INCI) |
| | ethyltrimethoxysilane | |
| 256 | (3-Aminopropyl)triethoxysilane + | Sulfated Rapeseed Oil (INCI) |
| | ethyltriethoxysilane | |
| 257 | (3-Aminopropyl)triethoxysilane + | Sulfated Rapeseed Oil (INCI) |
| | propyltrimethoxysilane | |
| 258 | (3-Aminopropyl)triethoxysilane + | Sulfated Rapeseed Oil (INCI) |
| | propyltriethoxysilane | |
| 259 | (3-Aminopropyl)triethoxysilane + | Sulfated Rapeseed Oil (INCI) |
| | hexyltrimethoxysilane | |
| 260 | (3-aminopropyl)triethoxysilane + | Sulfated Rapeseed Oil (INCI) |
| | hexyltriethoxysilane | |
| 261 | (3-Aminopropyl)triethoxysilane + | Sulfated Rapeseed Oil (INCI) |
| | octyltrimethoxysilane | |
| 262 | (3-Aminopropyl)triethoxysilane + | Sulfated Rapeseed Oil (INCI) |
| | Octyltriethoxysilane | |
| 263 | (3-Aminopropyl)triethoxysilane + | Sulfated Rapeseed Oil (INCI) |
| | dodecyltrimethoxysilane | |
| 264 | (3-Aminopropyl)triethoxysilane + | Sulfated Rapeseed Oil (INCI) |
| | dodecyltriethoxysilane | |
| 265 | (3-aminopropyl)triethoxysilane + | Sulfated Rapeseed Oil (INCI) |
| | octyldecyltrimethoxysilane | |
| 266 | (3-Aminopropyl)triethoxysilane + | Sulfated Rapeseed Oil (INCI) |
| | Octyldecyltriethoxysilane | |
| 267 | (3-Aminopropyl)triethoxysilane + | Sulfated Coconut Oil (INCI) |
| | methyltrimethoxysilane | |
| 268 | (3-Aminopropyl)triethoxysilane + | Sulfated Coconut Oil (INCI) |
| | methyltriethoxysilane | |
| 269 | (3-Aminopropyl)triethoxysilane + | Sulfated Coconut Oil (INCI) |
| | ethyltrimethoxysilane | |
| 270 | (3-Aminopropyl)triethoxysilane + | Sulfated Coconut Oil (INCI) |
| | ethyltriethoxysilane | |
| 271 | (3-Aminopropyl)triethoxysilane + | Sulfated Coconut Oil (INCI) |
| | propyltrimethoxysilane | |
| 272 | (3-Aminopropyl)triethoxysilane + | Sulfated Coconut Oil (INCI) |
| | propyltriethoxysilane | |
| 273 | (3-Aminopropyl)triethoxysilane + | Sulfated Coconut Oil (INCI) |
| | hexyltrimethoxysilane | |
| 274 | (3-aminopropyl)triethoxysilane + | Sulfated Coconut Oil (INCI) |
| | hexyltriethoxysilane | |
| 275 | (3-Aminopropyl)triethoxysilane + | Sulfated Coconut Oil (INCI) |
| | octyltrimethoxysilane | |
| 276 | (3-Aminopropyl)triethoxysilane + | Sulfated Coconut Oil (INCI) |
| | Octyltriethoxysilane | |
| 277 | (3-Aminopropyl)triethoxysilane + | Sulfated Coconut Oil (INCI) |
| | dodecyltrimethoxysilane | |
| 278 | (3-Aminopropyl)triethoxysilane + | Sulfated Coconut Oil (INCI) |
| | dodecyltriethoxysilane | |
| 279 | (3-aminopropyl)triethoxysilane + | Sulfated Coconut Oil (INCI) |
| | octyldecyltrimethoxysilane | |
| 280 | (3-Aminopropyl)triethoxysilane + | Sulfated Coconut Oil (INCI) |
| | Octyldecyltriethoxysilane | |
| 281 | (3-Aminopropyl)triethoxysilane + | sulfated soybean oil |
| | methyltrimethoxysilane | |
| 282 | (3-Aminopropyl)triethoxysilane + | sulfated soybean oil |
| | methyltriethoxysilane | |
| 283 | (3-Aminopropyl)triethoxysilane + | sulfated soybean oil |
| | ethyltrimethoxysilane | |
| 284 | (3-Aminopropyl)triethoxysilane + | sulfated soybean oil |
| | ethyltriethoxysilane | |
| 285 | (3-Aminopropyl)triethoxysilane + | sulfated soybean oil |
| | propyltrimethoxysilane | |
| 286 | (3-Aminopropyl)triethoxysilane + | sulfated soybean oil |
| | propyltriethoxysilane | |
| 287 | (3-Aminopropyl)triethoxysilane + | sulfated soybean oil |
| | hexyltrimethoxysilane | |
| 288 | (3-aminopropyl)triethoxysilane + | sulfated soybean oil |
| | hexyltriethoxysilane | |
| 289 | (3-Aminopropyl)triethoxysilane + | sulfated soybean oil |
| | octyltrimethoxysilane | |
| 290 | (3-Aminopropyl)triethoxysilane + | sulfated soybean oil |
| | Octyltriethoxysilane | |
| 291 | (3-Aminopropyl)triethoxysilane + | sulfated soybean oil |
| | dodecyltrimethoxysilane | |
| 292 | (3-Aminopropyl)triethoxysilane + | sulfated soybean oil |
| | dodecyltriethoxysilane | |
| 293 | (3-aminopropyl)triethoxysilane + | sulfated soybean oil |
| | octyldecyltrimethoxysilane | |
| 294 | (3-Aminopropyl)triethoxysilane + | sulfated soybean oil |
| | Octyldecyltriethoxysilane | |
| 295 | (3-Aminopropyl)triethoxysilane + | Sulfated Sunflower Seed Oil (INCI) |
| | methyltrimethoxysilane | |
| 296 | (3-Aminopropyl)triethoxysilane + | Sulfated Sunflower Seed Oil (INCI) |
| | methyltriethoxysilane | |
| 297 | (3-Aminopropyl)triethoxysilane + | Sulfated Sunflower Seed Oil (INCI) |
| | ethyltrimethoxysilane | |
| 298 | (3-Aminopropyl)triethoxysilane + | Sulfated Sunflower Seed Oil (INCI) |
| | ethyltriethoxysilane | |
| 299 | (3-Aminopropyl)triethoxysilane + | Sulfated Sunflower Seed Oil (INCI) |
| | propyltrimethoxysilane | |
| 300 | (3-Aminopropyl)triethoxysilane + | Sulfated Sunflower Seed Oil (INCI) |
| | propyltriethoxysilane | |
| 301 | (3-Aminopropyl)triethoxysilane + | Sulfated Sunflower Seed Oil (INCI) |
| | hexyltrimethoxysilane | |
| 302 | (3-aminopropyl)triethoxysilane + | Sulfated Sunflower Seed Oil (INCI) |
| | hexyltriethoxysilane | |
| 303 | (3-Aminopropyl)triethoxysilane + | Sulfated Sunflower Seed Oil (INCI) |
| | octyltrimethoxysilane | |
| 304 | (3-Aminopropyl)triethoxysilane + | Sulfated Sunflower Seed Oil (INCI) |
| | Octyltriethoxysilane | |
| 305 | (3-Aminopropyl)triethoxysilane + | Sulfated Sunflower Seed Oil (INCI) |
| | dodecyltrimethoxysilane | |
| 306 | (3-Aminopropyl)triethoxysilane + | Sulfated Sunflower Seed Oil (INCI) |
| | dodecyltriethoxysilane | |
| 307 | (3-aminopropyl)triethoxysilane + | Sulfated Sunflower Seed Oil (INCI) |
| | octyldecyltrimethoxysilane | |
| 308 | (3-Aminopropyl)triethoxysilane + | Sulfated Sunflower Seed Oil (INCI) |
| | Octyldecyltriethoxysilane | |
| 309 | (3-Aminopropyl)triethoxysilane + | Sulfated Olive Oil (INCI) |
| | methyltrimethoxysilane | |
| 310 | (3-Aminopropyl)triethoxysilane + | Sulfated Olive Oil (INCI) |
| | methyltriethoxysilane | |
| 311 | (3-Aminopropyl)triethoxysilane + | Sulfated Olive Oil (INCI) |
| | ethyltrimethoxysilane | |
| 312 | (3-Aminopropyl)triethoxysilane + | Sulfated Olive Oil (INCI) |
| | ethyltriethoxysilane | |
| 313 | (3-Aminopropyl)triethoxysilane + | Sulfated Olive Oil (INCI) |
| | propyltrimethoxysilane | |
| 314 | (3-Aminopropyl)triethoxysilane + | Sulfated Olive Oil (INCI) |
| | propyltriethoxysilane | |
| 315 | (3-Aminopropyl)triethoxysilane + | Sulfated Olive Oil (INCI) |
| | hexyltrimethoxysilane | |
| 316 | (3-aminopropyl)triethoxysilane + | Sulfated Olive Oil (INCI) |
| | hexyltriethoxysilane | |
| 317 | (3-Aminopropyl)triethoxysilane + | Sulfated Olive Oil (INCI) |
| | octyltrimethoxysilane | |
| 318 | (3-Aminopropyl)triethoxysilane + | Sulfated Olive Oil (INCI) |
| | Octyltriethoxysilane | |
| 319 | (3-Aminopropyl)triethoxysilane + | Sulfated Olive Oil (INCI) |
| | dodecyltrimethoxysilane | |
| 320 | (3-Aminopropyl)triethoxysilane + | Sulfated Olive Oil (INCI) |
| | dodecyltriethoxysilane | |
| 321 | (3-aminopropyl)triethoxysilane + | Sulfated Olive Oil (INCI) |
| | octyldecyltrimethoxysilane | |
| 322 | (3-Aminopropyl)triethoxysilane + | Sulfated Olive Oil (INCI) |
| | Octyldecyltriethoxysilane | |
| 323 | (3-Aminopropyl)triethoxysilane + | sulfated meadow foam oil |
| | methyltrimethoxysilane | |
| 324 | (3-Aminopropyl)triethoxysilane + | sulfated meadow foam oil |
| | methyltriethoxysilane | |
| 325 | (3-Aminopropyl)triethoxysilane + | sulfated meadow foam oil |
| | ethyltrimethoxysilane | |
| 326 | (3-Aminopropyl)triethoxysilane + | sulfated meadow foam oil |
| | ethyltriethoxysilane | |
| 327 | (3-Aminopropyl)triethoxysilane + | sulfated meadow foam oil |
| | propyltrimethoxysilane | |
| 328 | (3-Aminopropyl)triethoxysilane + | sulfated meadow foam oil |
| | propyltriethoxysilane | |
| 329 | (3-Aminopropyl)triethoxysilane + | sulfated meadow foam oil |
| | hexyltrimethoxysilane | |
| 330 | (3-aminopropyl)triethoxysilane + | sulfated meadow foam oil |
| | hexyltriethoxysilane | |
| 331 | (3-Aminopropyl)triethoxysilane + | sulfated meadow foam oil |
| | octyltrimethoxysilane | |
| 332 | (3-Aminopropyl)triethoxysilane + | sulfated meadow foam oil |
| | Octyltriethoxysilane | |
| 333 | (3-Aminopropyl)triethoxysilane + | sulfated meadow foam oil |
| | dodecyltrimethoxysilane | |
| 334 | (3-Aminopropyl)triethoxysilane + | sulfated meadow foam oil |
| | dodecyltriethoxysilane | |
| 335 | (3-aminopropyl)triethoxysilane + | sulfated meadow foam oil |
| | octyldecyltrimethoxysilane | |
| 336 | (3-Aminopropyl)triethoxysilane + | sulfated meadow foam oil |
| | Octyldecyltriethoxysilane | |

The combinations in the above table represent active ingredient combinations that are combined in cosmetic products with other components described above.

The active ingredient combination of at least one organic silicon compound and a sulfated and/or sulfonated fatty acid ester may already be present in the composition for treating a keratinous material. In this embodiment, the agent for treating a keratinous material is already distributed in a form ready for use. To provide a formulation that is as stable as possible during storage, the agent itself is preferably packaged with low or no water.

Alternatively, the at least one organic silicon compound is added a maximum of 12 hours, preferably a maximum of 6 hours, more preferably a maximum of 3 hours, even more preferably a maximum of 1 hour prior to application of the keratinous material treatment composition to a base comprising all the ingredients of the keratinous material treatment composition except the at least one organic silicon compound.

Furthermore, alternatively, the organic silicon compound and the sulfated/sulfonated fatty acid ester are added to a cosmetic product only shortly before application, i.e., from about 1 minute to about 12 hours, preferably from about 2 minutes to about 6 hours, particularly preferably from about 1 minute to about 3 hours, especially preferably from about 1 minute to about 1 hour.

In another alternative, the AMEO or the bis(triethoxysilylpropyl)amine is added to an aqueous solution, which is applied to the hair, and in the second step, an aqueous solution or a cosmetic agent containing the sulfated/sulfonated oil is applied to the hair.

For example, the user may first mix or shake an agent (α) comprising the organic silicon compound(s) with an agent (β) comprising the remaining ingredients of the agent for treating a keratinous material. The user can now apply this mixture of (α) and (β)—either directly after its preparation or after a short reaction time of about 1 minute to about 20 minutes-to the keratinous materials. The agent (β) may contain water, in particular water in an amount >30% by weight, based on the total weight of the agent for treating keratinous materials.

Another object of the present application is the use of a cosmetic composition as contemplated herein for treating a keratinous material for the care of keratinous material and/or for hydrophobizing the surface of keratinous material.

Regarding further preferred embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for treating a keratinous material comprising
a) at least one organic silicon compound containing one to three silicon atoms, and
b) at least one sulfated and/or sulfonated fatty acid ester.

2. A cosmetic composition for treating a keratinous material according to claim 1, wherein the at least one organic silicon compound containing one to three silicon atoms comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, two-band C₁-C₆-alkylene group,
R₃, R₄ independently represent a methyl group or an ethyl group,
a stands for the number 3 and
b stands for the number 0, and
wherein in the organic silicon compound of formula (II)
(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
(A″″)-Si(R₆″)_{d″}(OR₅″)_{c″} (III),
where
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the residues from e, f, g, and h is different from 0.

3. A cosmetic composition for treating a keratinous material according to claim 1,
wherein
that the agent for treating a keratinous material contains at least one organic silicon compound of formula (I) selected from the group consisting of
(3-Aminopropyl)trimethoxysilane,
(3-Aminopropyl)triethoxysilane,
(2-Aminoethyl)trimethoxysilane,
(2-Aminoethyl)triethoxysilane,
(3-Dimethylaminopropyl)trimethoxysilane,
(3-Dimethylaminopropyl)triethoxysilane,
(2-Dimethylaminoethyl)trimethoxysilane, and
(2-Dimethylaminoethyl)triethoxysilane,
or wherein,
the agent for treating a keratinous material contains at least one organic silicon compound of formula (II) selected from the group consisting of
3-(trimethoxysilyl)-N-[3-(trimethoxysilyl)propyl]-1-propanamine,
3-(Triethoxysilyl)-N-[3-(triethoxysilyl)propyl]-1-propanamine,
N-methyl-3-(trimethoxysilyl)-N-[3-(trimethoxysilyl)propyl]-1-propanamine,
N-Methyl-3-(triethoxysilyl)-N-[3-(triethoxysilyl)propyl]-1-propanamine,
2-[Bis[3-(trimethoxysilyl)propyl]amino]-ethanol,
2-[Bis[3-(triethoxysilyl)propyl]amino]ethanol,
3-(Trimethoxysilyl)-N,N-bis[3-(trimethoxysilyl)propyl]-1-propanamine,
3-(Triethoxysilyl)-N,N-bis[3-(triethoxysilyl)propyl]-1-propanamine,
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl)propyl]-2-propen-1-amine, and
N,N-Bis[3-(triethoxysilyl)propyl]-2-propen-1-amine.

4. A cosmetic composition for treating a keratinous material according to claim 1, wherein the organic silicon compound of formula (I) is present in the cosmetic composition in an amount of from about 0.01 to about 10% by weight, and/or characterized in that the organic silicon compound of formula (II) is present in an amount of from about 0.01 to about 10% by weight.
and/or wherein the organic silicon compound of formula (I) is (3-aminopropyl)triethoxysilane and/or the organic silicon compound of formula (II) is 3-(triethoxysilyl)-N-[3-(triethoxysilyl)propyl]-1-propanamine.

5. A cosmetic composition for treating a keratinous material according to claim 1, wherein the at least one sulfated and/or sulfonated fatty acid ester is a sulfated and/or sulfonated fatty acid triglyceride, preferably the sulfated and/or sulfonated fatty acid triglyceride forming a total amount in the cosmetic composition, the total amount of sulfated and/or sulfonated fatty acid triglyceride in the cosmetic composition being from about 0.1 to about 30% by weight.

6. Cosmetic composition for the treatment of a keratinous material according to claim 1, wherein the at least one sulphated and/or sulphonated fatty acid triglyceride in the cosmetic composition is a fatty acid triglyceride, the fatty acid groups of which are linear or branched, saturated or unsaturated, and/or hydroxy-functionalized, the fatty acid groups having 8 to 30.

7. A cosmetic composition for treating a keratinous material according to claim 1, wherein the composition for treating a keratinous material comprises at least one organic silicon compound of formula (IV),
R₉Si(OR₁₀)ₖ(R₁₁)ₘ (IV),
which is selected from the group consisting of:
Methyltrimethoxysilane;
Methyltriethoxysilane;
Ethyltrimethoxysilane;
Ethyltriethoxysilane;
Propyltrimethoxysilane;
Propyltriethoxysilane;
Hexyltrimethoxysilane;
Hexyltriethoxysilane;
Octyltrimethoxysilane;
Octyltriethoxysilane;
Dodecyltrimethoxysilane;
Dodecyltriethoxysilane;
Octyldecyltrimethoxysilane; and
Octyldecyltriethoxysilane.

8. A cosmetic composition for treating a keratinous material according to claim 1, wherein the sulfated and/or sulfonated fatty acid triglycerides are sulfated vegetable oils.

9. A cosmetic composition for treating a keratinous material according to claim 1, wherein the composition for treating a keratinous material comprises, based on the total weight of the composition for treating a keratinous material:
from about 0.5 to about 3% by weight of at least one first organic silicon compound selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7% by weight of at least one second organic silicon compound selected from the group consisting of methyltrimethoxysilane, methyltriethoxysilane, ethyltrimethoxysilane, ethyltriethoxysilane, propyltrimethoxysilane, propyltriethoxysilane, octyltrimethoxysilane, octyltriethoxysilane, dodecyltrimethoxysilane and dodecyltriethoxysilane.

10. Use of a cosmetic composition for treating a keratinous material according to claim 1.
for temporary reshaping of keratinous material,
for the care of keratinous material, and/or
for hydrophobizing the surface of keratinous material.

11. A cosmetic composition for treating a keratinous material according to claim 4, wherein the organic silicon compound of formula (I) is present in the cosmetic composition in an amount of from 0.1 to 4% by weight, based on the total weight of the cosmetic composition.

12. A cosmetic composition for treating a keratinous material according to claim 11, wherein the organic silicon compound of formula (I) is (3-aminopropyl)triethoxysilane.

13. A cosmetic composition for treating a keratinous material according to claim 4, wherein the organic silicon compound of formula (II) is present in an amount of from 0.1 to 6% by weight, based on the total weight of the cosmetic composition.

14. A cosmetic composition for treating a keratinous material according to claim 13, wherein the organic silicon compound of formula (II) is 3-(triethoxysilyl)-N-[3-(triethoxysilyl)propyl]-1-propanamine.

15. A cosmetic composition for treating a keratinous material according to claim 5, wherein the total amount of sulfated and/or sulfonated fatty acid triglyceride in the cosmetic composition is 1 to 10% by weight, based on the total weight of the cosmetic composition.

16. A cosmetic composition for treating a keratinous material according to claim 6, wherein the fatty acid groups have 12 to 24 C atoms.

17. A cosmetic composition for treating a keratinous material according to claim 8, wherein the sulfated vegetable oils are selected from sulfated rapeseed oil (INCI: Sulfated Rapeseed Oil), sulfated sunflower oil (INCI: Sulfated Sunflower Seed Oil), sulfated coconut oil (INCI: Sulfated Coconut Oil), sulfated castor oil (INCI: Sulfated Castor Oil), sulfated marsh flower oil, sulfated olive oil (INCI: Sulfated Olive Oil), and sulfated soybean oil.

18. A cosmetic composition for treating a keratinous material according to claim 8, wherein the sulfated vegetable oils are selected from sulfated coconut oil (INCI: Sulfated Coconut Oil), sulfated canola oil (INCI: Sulfated Rapeseed Oil) and/or sulfated castor oil (INCI: Sulfated Castor Oil).
